(19) United States
(12) Patent Application Publication
(10) Pub. No.: US 2003/0139810 A1
(43) Pub. Date: Jul. 24, 2003

(54) Methods for breast augmentation using balloon dissection and a rolled compressed inflatable implant

(76) Inventor: Melvyn Jay Bircoll, Los Angeles, CA (US)

Correspondence Address: MARK E. OGRAM, 7454 E. KOLB RD., STE. 203, TUCSON, AZ, 85710, US

(21) Appl. No.: 09/682,506

(22) Filed: Sep. 11, 2001

(51) Int. Cl. A61F 2/12

(52) U.S. Cl. 623/8; 623/902

(57) ABSTRACT

The invention discloses a method of breast augmentation that allows for (i) an umbilical or other distant incision, (ii) rapid, blunt dissection of a properly-sized, hemostatically-controlled retro-mammary pocket using a balloon dissector, and (iii) the placement of properly-sized inflatable implants that have been tested, compressed, rolled, sterilized and wrapped by the manufacturer (i.e., implants that are ready for insertion into the incision without further handling or manipulation by the surgeon).

### CROSS REFERENCE TO RELATED APPLICATIONS

Bircoll: U.S. Pat. No. 5,868,775. Feb. 9, 1999, Shaped Balloon Dissector and Implant Kit

### BACKGROUND OF INVENTION

Breast augmentation is a surgical procedure that has been performed successfully for many years. Traditionally, the procedure was performed with silicone gel prostheses. Recently, however, many doctors have abandoned the silicone gel prosthesis due to the medical-legal implications associated with it. Instead, these doctors use saline-filled inflatable prostheses in breast augmentation procedures. In the standard breast augmentation procedure, and regardless what kind of implant is actually used, an incision is made on or under the breast. This incision provides access to a retro-mammary space. In the retro-mammary space, the surgeon dissects a “pocket” and then places the implant inside that pocket. Prior to placing a saline-filled inflatable implant in the retro-mammary pocket he or she has created, the surgeon must remove the implant from its sterile container, fill the implant and test for the “integrity” of the implant (i.e., test to ascertain that there are no holes or other deformities in the implant) by squeezing it in its inflated state. Once the integrity of the implant has been confirmed, the surgeon empties the implant of all fluid and air, rolls the implant into a tightly-rolled “cigar-like” structure, and then forces the rolled implant, frequently with the aid of an instrument, into the pocket in the retro-mammary space. Next, the surgeon fills the implant with the desired quantity of saline through a filling tube with a self-sealing valve. Finally, the surgeon closes the incision and wraps the patient in a compression breast bandage.

The invention discloses a method of breast augmentation that allows for (i) an umbilical or other distant incision, (ii) rapid, blunt dissection of a properly-sized, hemostatically-controlled retro-mammary pocket using a balloon dissector, and finally (i.e., prior to completion of the procedure as follows), (iii) the placement of properly-sized inflatable implants that have been tested, compressed, rolled, sterilized and wrapped by the manufacturer (i.e., implants that are ready for insertion into the incision without further handling or manipulation by the surgeon). Once the implants have been inserted into the retro-mammary pocket, the surgeon may either close the distal (e.g., umbilical) incision, or leave it open for drainage. The surgeon then applies a dressing to the incision and puts the patient into a simple bra.

The method disclosed has several distinct advantages. First, but without limitation, the method invention enhances the safety of the breast augmentation procedure. Because the implants have been pre-prepared for insertion by the manufacturer, and the surgeon, therefore, does not have to perform the traditional tasks of testing and rolling the implants (the principal cause of damage to implants), there is a decreased likelihood of deflation of the implants and the resulting necessity of a secondary operation. Second, the method invention is a more economical way of performing breast augmentation. Not only does it permit the surgeon to perform the bilateral breast implant procedure in approximately 20 minutes (as opposed to the current 60 to 90 minutes), it is also more economical in that, as described above, it greatly reduces the necessity of secondary procedures. Secondary procedures are performed at great costs to all parties. The manufacturer must replace the implants without charge; the surgeon must perform the secondary procedure without charge; and the patient must undergo the secondary procedure. Third, the method invention enhances the satisfaction of the patient, not only by reducing the likelihood that the patient will have to undergo a secondary procedure, but also by allowing the patient to achieve excellent results, while undergoing a more minimally-invasive primary procedure that leaves no visible scar. Fourth, the method invention enhances the attractiveness to consumers of breast augmentation as a procedure generally insofar as it discloses a safer, more economical and patient-friendly alternative to current breast augmentation methods. Consequently, the method invention is certain to increase the popularity of breast augmentation and the number of breast augmentation procedures performed annually.

### DETAILED DESCRIPTION

The invention discloses a method of performing prosthetic breast augmentation using balloon dissection and a sterile implant rolled, compressed and placed into an insertion sheath by the manufacturer. The method invention procedure is performed as follows: The surgeon makes a small incision in a distal location (e.g., the umbilicus). The surgeon then inserts a balloon dissector with a suitably long insertion handle into the proper plane in the retro-mammary space. (Indices on the balloon dissector may guide proper orientation of the balloon dissector as it is being introduced into the retro-mammary space.) The surgeon then inflates the balloon dissector to a sufficient volume to permit (i) creation of an appropriately-sized “footprint” in the retro-mammary space and (ii) control of capillary bleeding. Next, the surgeon deflates the balloon dissector. He or she may also use the distal end of the balloon dissector to take down any remaining fibrous “bands”. The surgeon then removes the balloon dissector. After removing the balloon dissector, the surgeon may wish to check the retro-mammary pocket with the aid of an endoscope and to cauterize any remaining bleeding. Once the “dry” retro-mammary pocket has been created as described above, the surgeon is ready to insert the implants, without any further unfolding or handling of the implants.

The surgeon places a pre-wrapped, rolled and compressed (so as to be free of air) sterile implant, which is itself contained within an insertion sheath, and which together with the insertion sheath will have been prepared by the manufacturer, into the retro-mammary space through the distal (e.g., umbilical) incision. Indices on the implant and/or the insertion sheath may guide proper orientation of the implant/insertion sheath “complex” as it is being introduced into the retro-mammary space.

The surgeon then inflates the implant through a self-sealing filling tube to the desired volume. (Currently, the preferred inflation material is normal saline, but any liquid approved by the federal Food and Drug Administration for this purpose could be used with the method invention.) Next, the surgeon removes the self-sealing filling tube. The surgeon may then either close the distal (e.g., umbilical) incision in a conventional manner, or leave it open for safe, distal drainage. Finally, the surgeon applies a dressing to the incision and puts the patient into a simple bra.

Performance of the disclosed method is facilitated by a patented kit. See Bircoll, Shaped Balloon Dissector and Implant Kit, U.S. Pat. No. 5,868,775 (Feb. 9, 1999).

1. The invention discloses a method of performing prosthetic breast augmentation using balloon dissection and a sterile implant rolled, compressed and placed into an insertion sheath by the manufacturer. The method invention procedure is performed as follows: The surgeon makes a small incision in a distal location (e.g., the umbilicus). The surgeon then inserts a balloon dissector with a suitably long insertion handle into the proper plane in the retro-mammary space. (Indices on the balloon dissector may guide proper orientation of the balloon dissector as it is being introduced into the retro-mammary space.) The surgeon then inflates the balloon dissector to a sufficient volume to permit (i) creation of an appropriately-sized “footprint” in the retro-mammary space and (ii) control of capillary bleeding. Next, the surgeon deflates the balloon dissector. He or she may also use the distal end of the balloon dissector to take down any remaining fibrous “bands.” The surgeon then removes the balloon dissector. After removing the balloon dissector, the surgeon may wish to check the retro-mammary pocket with the aid of an endoscope and to cauterize any remaining bleeding. Once the “dry” retro-mammary pocket has been created as described above, the surgeon is ready to insert the implants, without any further unfolding or handling of the implants. The surgeon places a pre-wrapped, rolled and compressed (so as to be free of air) sterile implant, which is itself contained within an insertion sheath, and which together with the insertion sheath will have been prepared by the manufacturer, into the retro-mammary space through the distal (e.g., umbilical) incision. Indices on the implant and/or the insertion sheath may guide proper orientation of the implant/insertion sheath “complex” as it is being introduced into the retro-mammary space. The surgeon then inflates the implant through a self-sealing filling tube to the desired volume. (Currently, the preferred inflation material is normal saline, but any liquid approved by the federal Food and Drug Administration for this purpose could be used with the method invention.) Next, the surgeon removes the self-sealing filling tube. The surgeon may then either close the distal (e.g., umbilical) incision in a convention manner, or leave it open for safe, distal drainage. Finally, the surgeon applies a dressing to the incision and puts the patient into a simple bra.

2. Another embodiment of the invention discloses a method of performing prosthetic breast augmentation using balloon dissection and a sterile implant rolled, compressed and placed into an insertion sheath by the manufacturer, using a peri-areolar breast incision, an infra-mammary breast incision or an axillary incision.

3. Another embodiment of the invention discloses a method of performing prosthetic chin, cheek, calf or pectoral augmentation using balloon dissection and a sterile implant rolled, compressed and placed into an insertion sheath by the manufacturer.

4. Another embodiment of the invention discloses a method of performing prosthetic breast, cheek, calf and/or pectoral augmentation using balloon dissection and a sterile implant rolled, compressed and placed into an insertion sheath by the manufacturer, using a liquid material (other than normal saline) that has been approved by the federal Food and Drug Administration for the purpose of filling inflatable prosthetic implants.
